(19) United States
(12) Patent Application Publication
(10) Pub. No.: US 2024/0190717 A1
(43) Pub. Date: Jun. 13, 2024

(54) METHOD FOR PROMOTING THE RAPID PRECIPITATION OF TRAVERTINE CRYSTALS BY ALGAE

(71) Applicants: Chengdu University of Technology, Chengdu (CN); Jiangnan University, Wuxi (CN); Fudan University, Shanghai (CN)

(72) Inventors: Weizhen ZHANG, Chengdu (CN); Qi LI, Chengdu (CN); Ningfei LEI, Chengdu (CN); Jing ZHANG, Chengdu (CN); Guo LI, Chengdu (CN); Yu LIU, Chengdu (CN); Peng GU, Chengdu (CN); Xiaowei ZHENG, Chengdu (CN); Hanqi WU, Chengdu (CN); Wenhao GAO, Chengdu (CN); Jing LIU, Chengdu (CN); Yunxing XIAO, Chengdu (CN); Qihui LI, Chengdu (CN); Zheng ZHENG, Chengdu (CN)

(73) Assignees: Chengdu University of Technology, Chengdu (CN); Jiangnan University, Wuxi (CN); Fudan University, Shanghai (CN)

(21) Appl. No.: 18/105,890

(22) Filed: Feb. 6, 2023

(30) Foreign Application Priority Data
Dec. 8, 2022 (CN) 202211569342.6

(51) Int. Cl. C01F 11/18; C12N 1/12

(52) U.S. Cl. CPC C01F 11/181; C12N 1/12; C01P 2002/74

(57) ABSTRACT

A method for promoting the rapid precipitation of travertine crystals by algae is disclosed. Microalgae is added to a body of water having a calcium ion concentration of 100-500 mg/L and stirred. The amount of microalgae is 0.1-8×10⁸ cells/L. The invention adopts a method for promoting the rapid precipitation of travertine crystals by algae, which significantly improves the sedimentation rate of travertine crystals. At the same time, there are pseudomonas in the calcified water body of algae, which can be used for algae-lysing bacteria isolation and purification.

### CROSS REFERENCE TO THE RELATED APPLICATIONS

This application is based on and claims priority to Chinese Application No. 202211569342.6, filed Dec. 8, 2022, the entire contents of which are incorporated herein by reference.

### TECHNICAL FIELD

The invention relates to the field of water treatment technology, in particular to a method for promoting rapid precipitation of travertine crystals by algae.

### BACKGROUND

The main component of travertine is CaCO₃ precipitation. According to the water temperature of its formation place, travertine is divided into cold water type travertine and hot water type travertine. The formation of travertine is mainly the CO₂ overflow of the water body, which leads to the supersaturation of CaCO₃ in the water body, thus the precipitation is formed. The crystal structure of travertine is mainly calcite or aragonite. The main factors for travertine formation are the influence of geomorphic conditions, temperature and climate, hydrodynamic conditions, and biological effects. The current mainstream school believes that travertine is dominated by hydrodynamic factors. It is believed that under the influence of hydrodynamics, the water body emits CO₂, and the following equation occurs in the solution rich in Ca²⁺ and HCO³⁻: Ca²⁺+2HCO³⁻→CO₂↑+CaCO₃↓+H₂O, resulting in the precipitation of CaCO₃ in the solution, thus the landscape travertine is formed. However, in the scientific research of recent years, scholars Rogerson, M et al. and Shiraishi. F have simulated the sterilization of the water body without biological addition in the laboratory experiments, which only added the travertine system, and created the hydrodynamic conditions. However, there was no obvious travertine precipitation after the experiment, and a large amount of travertine precipitation was produced after adding the biomembrane, which proved that the biological factors are the main reason for the travertine formation.

Huanglong travertine landscape is mainly divided into the following four stages: the spring water rich in calcium ions overflows to form a thick layer of travertine sedimentations; the calcarenite sedimentation dissolution stage; the dynamic equilibrium stage of travertine sedimentation dissolution; the travertine degradation stage.

The travertine in Huanglong valley scenic spot has been degraded, such as the loose travertine surface layers, the travertine soil loss, and the blackening of travertine surface water. Due to the degradation of travertine in recent years, travertine conservation is extremely important. To conserve travertine and prevent travertine from degradation, it is of great significance to propose a method for rapid travertine sedimentation.

### SUMMARY

The purpose of this invention is to provide a method for promoting the rapid precipitation of travertine crystals by algae, which can significantly improve the sedimentation rate of travertine crystals. At the same time, there is pseudomonas in the calcified water body of algae, which can be used for algae-lysing bacteria isolation and purification.

To achieve the above purpose, the invention provides a method for promoting the rapid precipitation of travertine crystals by algae. Microalgae is added to the water body with a calcium ion concentration of 100-500 mg/L for stirring, and the amount of microalgae is 0.1-8×10⁸ cells/L.

The optimal selection is that the concentration of magnesium ion in water is 31.87 mg/L, and the concentration of bicarbonate ion is 796.8 mg/L.

The optimal selection is that the water pH is 7.5.

The optimal selection is that the concentration of calcium ion in water is 300 mg/L.

The optimal selection is that the added quantity of microalgae is 5.31×10⁷ cells/L.

The optimal selection is that the microalgae are one or several kinds of Chlorella and diatoms.

Therefore, the invention adopts a method for promoting the rapid precipitation of travertine crystals by algae, which significantly improves the sedimentation rate of travertine crystals. At the same time, there is pseudomonas in the calcified water body of algae, which can be used for algae-lysing bacteria isolation and purification.

A further detailed description of the technical solution of the invention through the drawings and embodiments is as follows.

### BRIEF DESCRIPTION OF THE DRAWINGS

FIG. 1 is the X-ray diffraction pattern;

FIGS. 2A-2C are SEM images of B1, C1, and D1;

FIGS. 3A-3F are SEM images of D1, D2, D3, D4, D5 and D6;

FIGS. 4A-4D are SEM images of A6, B6, C6, and D6.

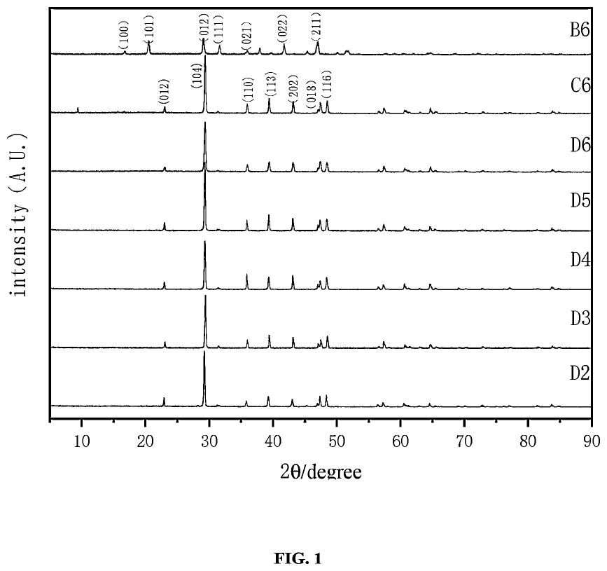

### DETAILED DESCRIPTION OF THE EMBODIMENTS

The technical solution of the invention is further explained by drawings and embodiments as follows.

### Embodiment 1

1) Prepare 5 portions of compound water with a calcium concentration of 100 mg/L.

2) Add the Chlorella into the compound water respectively. The adding quantities of Chlorella are 1.98×10⁷ cells/L, 5.31×10⁷ cells/L, 1.08×10⁸ cells/L, 4.86×10⁸ cells/L and 7.36×10⁸ cells/L, respectively.

3) Extract 15 ml water every two days for the detection of Ca²⁺ concentration change six times in total.

4) Calculate the sedimentation rate of travertine crystals in compound water by the exponential decay equation. The results are shown in Table 1.

TABLE 1
The sedimentation rate of travertine crystal in compound
water when the Ca²⁺ concentration is 100 mg/L

| Ca²⁺ initial | The adding quantities | The sedimentation |
|---|---|---|
| concentration mg/L | of Chlorella mg/L | rate mg/L · d |
| 100 | 1.98 × 10⁷ | 0.11010 |
| 100 | 5.31 × 10⁷ | 0.15535 |
| 100 | 1.08 × 10⁸ | 0.11621 |
| 100 | 4.86 × 10⁸ | 0.06961 |
| 100 | 7.36 × 10⁸ | 0.03735 |

### Embodiment 2

1) Prepare 5 portions of compound water with a calcium concentration of 300 mg/L.

2) Add the Chlorella into the compound water respectively. The adding quantities of Chlorella are 1.98×10⁷ cells/L, 5.31×10⁷ cells/L, 1.08×10⁸ cells/L, 4.86×10⁸ cells/L and 7.36×10⁸ cells/L, respectively.

3) Extract 15 ml water every two days for the detection of Ca²⁺ concentration change six times in total.

4) Calculate the sedimentation rate of travertine crystals in compound water by the exponential decay equation. The results are shown in Table 2.

TABLE 2
The sedimentation rate of travertine crystal in compound
water when the Ca²⁺ concentration is 300 mg/L

| Ca²⁺ initial | The adding quantities | The sedimentation |
|---|---|---|
| concentration mg/L | of Chlorella mg/L | rate mg/L · d |
| 300 | 1.98 × 10⁷ | 0.14290 |
| 300 | 5.31 × 10⁷ | 0.22397 |
| 300 | 1.08 × 10⁸ | 0.18120 |
| 300 | 4.86 × 10⁸ | 0.16235 |
| 300 | 7.36 × 10⁸ | 0.14096 |

### Embodiment 3

1) Prepare 5 portions of compound water with a calcium concentration of 500 mg/L.

2) Add the Chlorella into the compound water respectively. The adding quantities of Chlorella are 1.98×10⁷ cells/L, 5.31×10⁷ cells/L, 1.08×10⁸ cells/L, 4.86×10⁸ cells/L and 7.36×10⁸ cells/L, respectively.

3) Extract 15 ml water every two days for the detection of Ca²⁺ concentration change six times in total.

4) Calculate the sedimentation rate of travertine crystals in compound water by the exponential decay equation. The results are shown in Table 3.

TABLE 3
The sedimentation rate of travertine crystal in compound
water when the Ca²⁺ concentration is 500 mg/L

| Ca²⁺ initial | The adding quantities | The sedimentation |
|---|---|---|
| concentration mg/L | of Chlorella mg/L | rate mg/L · d |
| 500 | 1.98 × 10⁷ | 0.04465 |
| 500 | 5.31 × 10⁷ | 0.04032 |
| 500 | 1.08 × 10⁸ | 0.06626 |
| 500 | 4.86 × 10⁸ | 0.07402 |
| 500 | 7.36 × 10⁸ | 0.08405 |

### Comparison Case 1

1) Prepare 5 portions of compound water with a calcium concentration of 100 mg/L.

2) Proceed with the natural sedimentation.

3) Extract 15 ml water every two days for the detection of Ca²⁺ concentration change six times in total.

4) Calculate the sedimentation rate of travertine crystals in compound water by the exponential decay equation. The results are shown in Table 4.

### Comparison Case 2

1) Prepare 5 portions of compound water with a calcium concentration of 300 mg/L.

2) Proceed with the natural sedimentation.

3) Extract 15 ml water every two days for the detection of Ca²⁺ concentration change six times in total.

4) Calculate the sedimentation rate of travertine crystals in compound water by the exponential decay equation. The results are shown in Table 4.

### Comparison Case 3

1) Prepare 5 portions of compound water with a calcium concentration of 500 mg/L.

2) Proceed with the natural sedimentation.

3) Extract 15 ml water every two days for the detection of Ca²⁺ concentration change six times in total.

4) Calculate the sedimentation rate of travertine crystals in compound water by the exponential decay equation. The results are shown in Table 4.

TABLE 4
The sedimentation rate of travertine crystal
in compound water (Comparison cases 1-3)

| Ca²⁺ initial | The adding quantities | The sedimentation |
|---|---|---|
| concentration mg/L | of Chlorella mg/L | rate mg/L · d |
| 100 | 0 | 0.03768 |
| 300 | 0 | 0.05049 |
| 500 | 0 | 0.01904 |

### The Ca²⁺ Concentration Testing Method

Extract 15 mL water and measure the Ca²⁺ concentration by ICP. The exponential decay equation is used to fit the process of travertine sedimentation. The exponential decay equation is expressed by equation 1, and the exponential decay equation after the logarithm is expressed by equation 2.

[Ca²⁺]=e^{kt+b} (1)

In[Ca²⁺]=kt+b (2)

Where [Ca²⁺] represents the calcium ion concentration, t represents the sampling time, k represents the travertine rate constant, d[Ca²⁺]/dt represents the travertine rate.

### The Travertine Morphology Testing Method

After 15 days, the deposited travertine crystals in the experiment were detected by XRD and SEM.

### The Analysis of Experimental Results

### 1. The Effect of Different Concentrations of Microalgae on the Calcification Rate: According to the Results of the Sedimentation Rate of Travertine Crystals in Compound Water in Table 1-4,

When the initial concentration of Ca²⁺ is 100.00 mg/L or 300.00 mg/L, the sedimentation rate of travertine crystals was the fastest in the compound water of microalgae solution with the initial concentration of 5.31×10⁷ cells/L. When the initial concentration of Ca²⁺ was 500.00 mg/L, the calcification rate went faster as the increase of microalgae concentration. The sedimentation rate of travertine crystals in comparison cases 1-3 is significantly lower than that of travertine crystals in embodiments 1-3. At different microalgae concentrations, when the Ca²⁺ concentration in the water was 300.00 mg/L, the calcification rate is the fastest.

Therefore, in low and medium concentrations of Ca²⁺ water, the calcification rate is the fastest when the initial concentration of microalgae solution is 5.31×10⁷ cells/L. At this time, when the concentration of microalgae increases, the calcification rate decreases instead. In high concentrations of Ca²⁺ water, the increase of microalgae concentration will increase the calcification rate. When the concentration of Ca²⁺ is about 300.00 mg/L, it is beneficial to increase the sedimentation rate of travertine crystals in water when the concentration of microalgae is about 5.31×10⁷ cells/L.

### 2. The Effect of Microalgae on the Travertine Crystal Structure

Samples of embodiments 1-3 and comparison cases 1-3 are numbered, as shown in Table 5, and analyzed by XRD. The X-ray diffraction (XRD) cell parameters of travertine sedimentation are shown in table 6.

TABLE 5
Sample number table

| | Ca²⁺ initial | Microalgae initial | |
|---|---|---|---|
| | concentration mg/L | concentration cells/L | No. |
| Comparison case 1 | 100 | 0 | B1 |
| Comparison case 2 | 200 | 0 | C1 |
| Comparison case 3 | 300 | 0 | D1 |
| Embodiment 1 | 100 | 1.98 × 10⁷ | B2 |
| | 100 | 5.31 × 10⁷ | B3 |
| | 100 | 1.08 × 10⁸ | B4 |
| | 100 | 4.86 × 10⁸ | B5 |
| | 100 | 7.36 × 10⁸ | B6 |
| Embodiment 2 | 300 | 1.98 × 10⁷ | C2 |
| | 300 | 5.31 × 10⁷ | C3 |
| | 300 | 1.08 × 10⁸ | C4 |
| | 300 | 4.86 × 10⁸ | C5 |
| | 300 | 7.36 × 10⁸ | C6 |
| Embodiment 3 | 500 | 1.98 × 10⁷ | D2 |
| | 500 | 5.31 × 10⁷ | D3 |
| | 500 | 1.08 × 10⁸ | D4 |
| | 500 | 4.86 × 10⁸ | D5 |
| | 500 | 7.36 × 10⁸ | D6 |

TABLE 6
The X-ray diffraction (XRD) cell parameters of travertine sedimentation

| Group | | D2 | D3 | D4 | D5 | D6 | C6 | B6 | |
|---|---|---|---|---|---|---|---|---|---|
| The chemical | | CaCO₃ | | | | | | CaCO₃ (H₂0) | |
| formula | | | | | | | | | |
| The crystal | | Hexagonal, calcite structure | | | | | | The hexagonal system, | |
| structure | | | | | | | | calcite monohydrate | |
| | | | | | | | | structure | |
| The space group | | R-3C(167) | | | | | | p3121 (152) | |
| The crystal | a/nm | 0.49896 | 0.49896 | 0.49880 | 0.49890 | 0.49890 | 0.49890 | a/nm | 0.60931 |
| cell refinement | b/nm | 0.49896 | 0.49896 | 0.49880 | 0.49890 | 0.49890 | 0.49890 | b/nm | 0.60931 |
| parameters | c/nm | 1.70610 | 1.70610 | 1.70680 | 1.70620 | 1.70620 | 1.70620 | c/nm | 0.75446 |
| | α/° | 90 | | | | | | | |
| | β/° | 90 | | | | | | | |
| | γ/° | 120 | | | | | | | |
| Cell | | 0.36785 | 0.36785 | 0.36776 | 0.36778 | 0.36778 | 0.36778 | Cell volume | 0.24292 |
| volume/nm³ | | | | | | | | | |
| Cell | | 2.71080 | 2.71080 | 2.71140 | 2.71120 | 2.71120 | 2.71120 | Cell density | 2.42530 |
| density(g/cm³) | | | | | | | | | |
| Number of unit | | 6 | | | | | | 3 | |
| cell molecules | | | | | | | | | |
| The main | \textcircled{1} (012) | 22.9270 | 23.0510 | 22.9640 | 22.9910 | 23.0500 | 23.0090 | \textcircled{1} (100) | 16.7910 |
| advantages | \textcircled{2} (104) | 29.2400 | 29.4110 | 29.3220 | 29.3200 | 29.3610 | 29.3590 | \textcircled{2} (101) | 20.5280 |
| correspond | \textcircled{3} (110) | 35.8110 | 35.9810 | 35.8930 | 35.9140 | 35.9820 | 35.9560 | \textcircled{1} (012) | 29.0620 |
| to2θ/° | \textcircled{4} (113) | 39.2590 | 39.4120 | 39.3300 | 39.3430 | 39.3930 | 39.3960 | \textcircled{2} (111) | 31.6220 |
| | \textcircled{5} (202) | 43.0250 | 43.1530 | 43.1060 | 43.1130 | 43.1390 | 43.1490 | \textcircled{3} (021) | 35.9620 |
| | \textcircled{6} (018) | 47.3640 | 47.5110 | 47.4010 | 47.3700 | 47.4030 | 47.4190 | \textcircled{4} (022) | 41.7250 |
| | \textcircled{7} (116) | 48.3650 | 48.5280 | 48.4100 | 48.4290 | 48.4440 | 48.4570 | \textcircled{5} (211) | 47.0260 |
| Cell | \textcircled{1} (012) | 0.38757 | 0.38522 | 0.38695 | 0.38651 | 0.38553 | 0.38622 | \textcircled{1} (100) | 0.52756 |
| Spacing | \textcircled{2} (104) | 0.30517 | 0.30344 | 0.30434 | 0.30436 | 0.30395 | 0.30397 | \textcircled{2} (101) | 0.43230 |
| | \textcircled{3} (110) | 0.25054 | 0.24940 | 0.24998 | 0.24985 | 0.24939 | 0.24956 | \textcircled{3} (012) | 0.30701 |
| | \textcircled{4} (113) | 0.22929 | 0.22844 | 0.22889 | 0.22882 | 0.22854 | 0.22852 | \textcircled{4} (111) | 0.28270 |
| | \textcircled{5} (202) | 0.21005 | 0.20946 | 0.20968 | 0.20965 | 0.20942 | 0.20948 | \textcircled{5} (021) | 0.24952 |
| | \textcircled{6} (018) | 0.19178 | 0.19121 | 0.19163 | 0.19175 | 0.19163 | 0.19156 | \textcircled{6} (022) | 0.21630 |
| | \textcircled{7} (116) | 0.18804 | 0.18744 | 0.18787 | 0.18780 | 0.18775 | 0.18770 | \textcircled{7} (211) | 0.19307 |
| Grain size | \textcircled{1} (012) | >100 | >100 | 76.5 | 86.9 | 61.7 | 79.4 | \textcircled{1} (100) | 59.5 |
| corresponding | \textcircled{2} (104) | 83.9 | 89.8 | 63.3 | 84.9 | 49.1 | 78.6 | \textcircled{2} (101) | 44.5 |
| to main | \textcircled{3} (110) | 73.6 | >100 | >100 | >100 | 53.4 | 81.5 | \textcircled{3} (012) | 46.1 |
| advantage | \textcircled{4} (113) | 69.4 | >100 | 93.6 | >100 | 56.1 | 97.8 | \textcircled{4} (111) | 48.2 |
| surface/nm | \textcircled{5} (202) | >100 | >100 | >100 | >100 | 52.8 | 77.5 | \textcircled{5} (021) | 60.4 |
| | \textcircled{6} (018) | >100 | >100 | 92.7 | 89.8 | 56.4 | 77.2 | \textcircled{6} (022) | 45.3 |
| | \textcircled{7} (116) | >100 | >100 | >100 | 79.0 | 65.2 | 97.7 | \textcircled{7} (211) | 29.6 |

| | | | | | | | | |
|---|---|---|---|---|---|---|---|---|
| Average grain | 68.6 | 75.4 | 61.8 | 71.9 | 43.2 | 65.7 | Average | 25.0 |
| size/nm | | | | | | | size/nm | |

It can be seen from the unit cell parameter table that in the experimental groups of D2, D3, D4, D5, D6, and C6, the fresh travertine contains the characteristic diffraction peaks of calcite (the main chemical composition is CaCO₃), and the corresponding main advantage surfaces are (012), (104), (110), (113), (202), (018) and (116). The space groups of D2, D3, D4, D5, D6, and C6 are all R-3c (167), the number of unit cell molecules is 6, the 20 corresponding to the main advantage surface is similar, and the cell spacing corresponding to the main advantage surface is not much different. The cell volume is 0.36785 nm³, 0.36776 nm³, and 0.36778 nm³. The cell density is 2.71080 g/cm³, 2.71120 g/cm³, and 2.71140 g/cm³. The average grain size of travertine crystal is less than 100.

The grain sizes corresponding to the main advantage surfaces of D2, D3, D4, and D5 are partially greater than 100, and the grain sizes corresponding to the main advantage surfaces of D6 and C6 are less than 100.

The B6 fresh travertine contains calcite characteristic diffraction peaks (the main chemical composition is CaCO₃(H₂O), and the corresponding main advantage surfaces are (100), (101), (012), (111), (021), (022), (211), etc. The B6 space group is P3121 (152), and the number of unit cell molecules is 3. The 20 corresponding to the main advantage surface and the cell spacing corresponding to the main advantage surface are different from those of the D2, D3, D4, D5, D6, and C6 groups. The cell volume is 0.24292 nm³, the cell density is 2.42530 g/cm³, and the average grain size of the travertine crystal is 25.0. The grain size corresponding to the main advantage surface of B⁶ is less than 100.

The XRD results show that at the same calcium ion concentration, the travertine crystals produced by different concentrations of microalgae have the same structure as calcite. When the concentration of microalgae increases to 7.36×10⁸ cells/L, the cell size of calcite decreases. The results of calcine sedimentation in water with different Ca²⁺ concentrations under the same microalgae concentration showed that when the Ca²⁺ concentration in the solution decreases to 79.68 mg/L, the structure of calcite will change and may become calcite monohydrate (CaCO₃·H₂O).

### 3. The Effect of Microalgae on Travertine Crystal Morphology

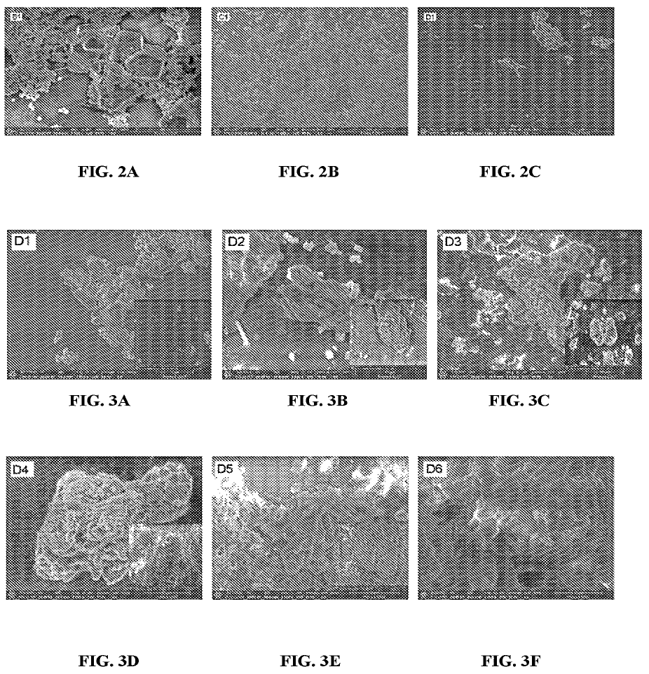

By observing the electron microscope images of the blank group without microalgae solution under different Ca²⁺ concentrations (as shown in FIGS. 2A-2C):

When Ca²⁺ is 100.00 mg/L, the calcite travertine is observed, but at this time, it is mainly in small crystal travertine form with a small amount of standard calcite.

When Ca²⁺ concentration increases to 300.00 mg/L, the proportion of small crystal travertine increases.

When the concentration of Ca²⁺ increases to 500.00 mg/L, the size of small crystal travertine increases, and filamentous algae imprinting is observed.

Comparing the D2-D6 experimental groups with different microalgae concentrations at the same calcium ion concentration (500.00 mg/mL), it was found that when the microalgae concentration increases, the shape of the formed travertine will be closer to the standard and mature calcite, and at the same time, due to the high concentration of microalgae, its dead body was covered on the surface of the travertine crystal, resulting in only a very small amount of travertine seen in the electron microscope picture. In the case of low-concentration microalgae, more travertine crystals can be observed by scanning electron microscope. However, other salt crystals appeared in the lowest concentration of microalgae solution.

By observing the electron microscope images of the experimental groups with different Ca²⁺ concentrations under the same microalgae concentration (as shown in FIGS. 3A-3F), it was found that the blank group (A6) with Ca²⁺ equals to 0 had no travertine crystals, only the other salt crystals appeared. The electron microscopy images of the experimental group showed that with the increase of calcium ion concentration, the travertine crystals developed more mature, and the travertine crystal contour became clear (calcite with unclear contour-petal shape calcite-hexagonal calcite).

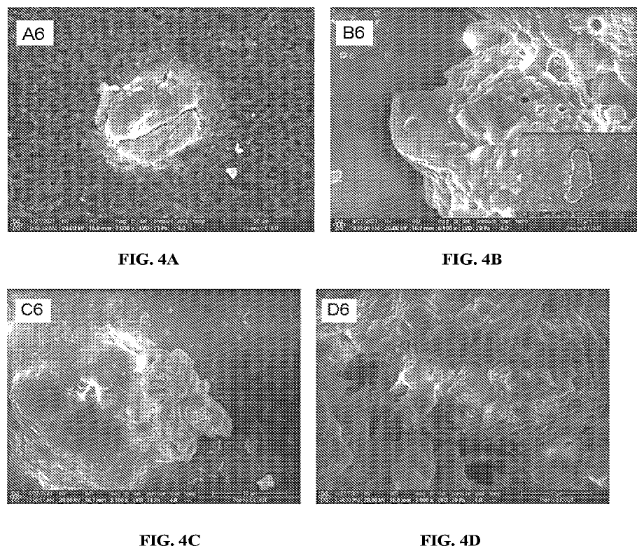

By observing all samples (FIGS. 2A-2C, 3A-3F, and 4A-4D), it can be found that the travertine sedimentation is based on the pores of microalgae. The travertine produced by microalgae produces microalgae accumulation first, then the travertine is generated based on microalgae accumulation, and then the microalgae accumulation covers the travertine. This process will occur repeatedly in the process of microalgae sedimentation. This phenomenon is the crystal core of microalgae calcic sedimentation.

In summary, this method can be used to repair travertine in degraded or damaged areas of the travertine landscape by cultivating microalgae.

Finally, it should be noted that the above embodiments are only used to explain the technical solution of the invention rather than to limit it. Although the invention is described in detail with references to the better embodiments, ordinary technicians in this field should understand that they can still modify or replace the technical solution of the invention, and these modifications or equivalent replacements cannot make the modified technical solution out of the spirit and scope of the technical solution of the invention.

What is claimed is:

1. A method for promoting a rapid precipitation of travertine crystals by an algae comprising adding a microalgae to a body of water having a calcium ion concentration of 100-500 mg/L to obtain a resulting mixture and stirring the resulting mixture, wherein an amount of the microalgae is 0.1-8×10⁸ cells/L.

2. The method for promoting the rapid precipitation of the travertine crystals by the algae according to claim 1, wherein a concentration of a magnesium ion in the water is 31.87 mg/L, and a concentration of a bicarbonate ion in the water is 796.8 mg/L.

3. The method for promoting the rapid precipitation of the travertine crystals by the algae according to claim 2, wherein a pH of the water is 7.5.

4. The method for promoting the rapid precipitation of the travertine crystals by the algae according to claim 1, wherein the calcium ion concentration in the water is 300 mg/L.

5. The method for promoting the rapid precipitation of the travertine crystals by the algae according to claim 1, wherein an added quantity of the microalgae is 5.31×10⁷ cells/L.

6. The method for promoting the rapid precipitation of the travertine crystals by the algae according to claim 1, wherein the microalgae is at least one selected from the group consisting of Chlorella and diatoms.
